(19) United States
(12) Patent Application Publication
(10) Pub. No.: US 2003/0175810 A1
(43) Pub. Date: Sep. 18, 2003

(54) Assay method and system for identification of P2Y receptor agonists and antagonists

(76) Inventors: T. Kendall Harden, Carrboro, NC (US); Gary L. Waldo, Chapel Hill, NC (US); Rainer Blaesius, Chapel Hill, NC (US); Robert Nicholas, Chapel Hill, NC (US)

Correspondence Address: JENKINS & WILSON, PA, 3100 TOWER BLVD, SUITE 1400, DURHAM, NC, 27707, US

(21) Appl. No.: 10/336,608

(22) Filed: Jan. 3, 2003

Related U.S. Application Data
(63) Continuation of application No. PCT/US01/21467, filed on Jul. 6, 2001.
(60) Provisional application No. 60/216,618, filed on Jul. 7, 2000.

(51) Int. Cl. G01N 33/53; C07H 21/04; C07K 14/705; C12P 21/02; C12N 5/06

(52) U.S. Cl. 435/7.1; 435/69.1; 435/320.1; 435/348; 530/350; 536/23.5

(57) ABSTRACT

A method and system for assaying P2Y receptors. The method allows for direct detection of ligand binding events with a high signal to noise ratio. The ability to quantify binding events involving the P2Y receptor has heretofore been unavailable due to unavoidable non-selective binding of a radioligand or other traditional probes. A cell-free system for the study of P2Y receptors including a P2Y receptor; optionally, a protein that is normally associated with the P2Y receptor in nature; and a vesicle, is also disclosed.

### GRANT STATEMENT

This work was supported by National Institutes of Health (NIH) grant NIGMS 38213. Thus, the U.S. Government has certain rights in the invention.

### TECHNICAL FIELD

The present invention relates generally to an assay method and system for identification of nucleotide binding protein agonists and antagonists. More particularly, the present invention relates to an assay method and system for identification of P2Y-receptor agonists and antagonists.

Table of Abbreviations

| | |
|---|---|
| ATP | adenosine 5′-triphosphate |
| ADP | adenosine 5′-diphosphate |
| FLAG°® | epitope comprising the amino acid sequence: |
| | Asp-Tyr-Lys-Asp-Asp-Asp-Asp-Lys (SEQ ID |
| | NO:1) |
| G protein | guanadine nucleotide-binding protein |
| GAP | GTPase activating protein |
| GPCR | G-protein coupled receptor |
| kDa | kilodalton (s) |
| 2MeSATP | 2-methylthioadenosine 5′-triphosphate |
| 2MeSADP | 2-methylthioadenosine 5′-diphosphate |
| NDP | nucleotide 5′-diphosphate |
| NTP | nucleotide 5′-triphosphate |
| NTPase | nucleotide 5′-triphosphatase |
| P2 | family of nucleotide receptors that includes the |
| | P2X and P2Y receptor subgroups |
| P2X | ionotropic, ATP-activated, ligand-gated ion |
| | channel-type receptor |
| P2Y | G protein coupled receptor for extracellular |
| | nucleotides that have been shown to be |
| | functional receptors |
| P2Y₁ | P2Y receptor strongly activated by 2-methylthio- |
| | ATP and ATP |
| P2Y₁-R | P2Y₁ receptor |
| P2Y₂ | P2Y receptor that binds both ATP and UTP, |
| | originally called P2U |
| PC | phosphatidyl choline |
| PCR | polymerase chain reaction |
| PE | phosphatidyl ethanolamine |
| PG | phosphatidyl glycerol |
| PI | phosphatidyl inositol |
| PKC | protein kinase C |
| PLC | phospholipase C |
| PMSF | phenylmethylsulfonyl fluoride |
| PS | phosphatidyl serine |
| RGS | regulator (protein) of G protein signaling |
| RhoA | a small GTP-binding protein that controls |
| | reorganization of the actin cytoskeleton and |
| | activates transcription factors in response to |
| | extracellular agonists |
| TPCK | N-tosyl-L-phenylalanine chloromethyl ketone |
| UTP | uridine 5′-triphosphate |

### BACKGROUND ART

Nucleotides are ubiquitous extracellular signaling molecules that give rise to a wide spectrum of biological responses that are mediated by P2 receptors. Since 1993 and 1994, when the first P2 receptors were cloned, the P2 receptors have been divided into two families: ionotropic P2X receptors, and metabotropic P2Y receptors. The latter are included in the superfamily of G-protein-coupled receptors. P2Y₁ and P2Y₂ were the first P2Y receptors to be cloned and closely correspond with earlier characterized subtypes (P2Y, P2U). Since 1994, homology cloning has isolated several new receptor subtypes.

Extracellular nucleotides control a wide variety of physiological responses by interacting with two types of cell surface P2 receptors (Fredholm et al. (1997) Drug Dev. Res. 39: 461-66). As noted, the P2X receptors are ionotropic and are ATP-activated P2X ligand-gated ion channels. Seven members of the P2X class of signaling proteins have been identified. In addition, a P2Y G protein-coupled receptor (GPCR) (Lustig et al. (1992) Biochim. Biophys. Acta 1134: 61-72) family has been identified. There are at least five known P2Y receptor subtypes in mammals (Fredholm et al. (1997) Trends Pharmacol. Sci. 18:79-82). P2Y subtypes have been classified pharmacologically and molecularly, and are predominantly linked to activation of phospholipase C (PLC) and increased levels of inositol 1,4,5-trisphosphate and diacylglycerol. This condition can lead to elevations in intracellular free calcium concentration ([Ca²⁺]ᵢ) and the activation of protein kinase C (PKC) (Lustig et al. (1992) Biochim. Biophys. Acta 1134: 61-72; Pearce et al. (1989) J. Neurochem. 52: 971-77; Sasakawa et al. (1989) J. Neurochem. 52: 441-47; Lin et al. (1993) J. Neurochem. 60: 1115-25). Specifically, the P2Y receptor subfamily includes the P2Y₁ receptor, which is activated by adenine nucleotides (Webb et al. (1993) FEBS Lett. 324: 219-25; Schachter et al. (1996) Br. J. Pharmacol. 118: 167-73); the P2Y₂ receptor, which is activated equipotently by ATP and UTP (Lustig et al. (1993) Proc. Natl. Acad. Sci. USA 90: 5113-17); the P2Y₄ receptor, which is potently activated by UTP (Communi et al. (1995) J. Biol. Chem. 270: 30849-52; Nguyen et al. (1995) J. Biol. Chem. 270: 30845-48); the P2Y₆ receptor, which is selectively activated by UDP (Chang et al. (1995) J. Biol. Chem. 270: 26152-58; Nicholas et al. (1996) Mol. Pharmacol. 50: 224-29); and the P2Y₁₁, receptor, which is a selective purinoreceptor and is dually coupled to both PLC and adenylate cyclase stimulation (Communi et al. (1999) Brit. J. Pharmacol. 128: 6 1199-206; Boeynaems etal. (2000) Trends Pharmacol. Sci. 21:1-3; WO 99/02675A1).

As noted, P2Y receptors are G protein-coupled receptors that are activated by extracellular nucleotides (Fredholm et al. (1994) Pharmacol. Rev. 46: 143-56). The family of G protein-coupled receptors, including the P2Y subfamily, is a group of membrane-associated proteins and exhibit a common topology and properties. Each member of the P2Y receptor subfamily comprises seven transmembrane helices with a short N-terminal domain at the extracellular surface. The P2Y subfamily of receptors responds to different degrees when exposed to extracellular adenine and uridine nucleotides. Sequence comparisons between P2Y and adenosine receptors have revealed several positively charged amino acid residues in transmembrane regions 3, 6 and 7 of the P2Y receptors (Boarder et al (1995) Trends Pharmacol. Sci. 16:133-39), leading to the suggestion that these residues are involved in the binding of negatively charged phosphate moieties presented by P2 receptor agonists. The exact nature of P2Y/G-protein interaction is not well understood, however effector activation suggests coupling to multiple G-proteins, including the Gₒ, Gᵢ and G_{q}/11 proteins (Boarder et al. (1995) Trends Pharmacol. Sci. 16:133-39; Dubyak et al. (1996) Drug Dev. Res. 39: 269-78; Harden et al (1996) Ann. Rev. Pharmacol. Toxicol. 36: 429-59).

Progress in this field of study has been difficult because there is a noticeable lack of accurate receptor binding data available to researchers and clinicians. Direct ligand binding data for the P2Y family of receptors, in particular, are lacking. This has followed from: (1) the lack of availability of suitable high affinity ligands; (2) the low levels of receptors in most cells; and (3) the large number of non-receptor proteins that bind nucleotides with high affinity and specificity.

The absence of a reliable binding assay for the P2Y receptor family has led to the development of a series of indirect assays. Most of these assays are based on the activation of various downstream signaling responses to ligand interactions at the P2Y receptor interface, but do not directly address receptor-ligand interactions. An additional problem with current assay systems and models is that they can be compromised by agonist-induced receptor desensitization.

As mentioned above, a problem with current assay systems and models for the study of extracellular nucleotides is that they cannot take into account the presence of the range of ectoenzymes normally present in laboratory model systems. Existent ectoenzymes can convert triphosphates to diphosphates and monophosphates via nucleotidases (Zimmerman (1996), Drug Dev. Res. 39:337-52), and triphosphates can be formed from diphosphates by nucleotide diphosphokinases (Lazarowski et al (1997), J. Biol. Chem. 272: 24348-54). These effects lead to inaccurate binding and kinetic data. The problems surrounding modification of NTPs cannot, therefore, be solved by simply using highly purified stock solutions of NTPs to be tested, because similar molecules can arise, by action of ectoenzymes, as metabolites during measurements of signaling responses.

What is needed, therefore, is a reliable and sensitive binding assay for P2Y receptors. Such an assay would: (1) address the problems normally associated with the ligand binding assays now in use; (2) substantially eliminate the potential for modification of applied extracellular nucleotides and thereby give accurate binding data; and (3) allow direct observation of ligand binding, and thereby eliminate the need to extrapolate information about ligand binding events from data taken by monitoring secondary messenger and other indirect effects. Such an assay is not available in the art.

### SUMMARY OF THE INVENTION

A method of screening candidate substances for an ability to modulate P2Y receptor-mediated biological activity is disclosed. The method comprises: (a) establishing a test sample comprising a substantially pure P2Y receptor; (b) contacting the test sample with a candidate substance; and (c) measuring an interaction, effect, or combination thereof, of the candidate substance on the test sample to thereby determine the ability of the candidate substance to modulate P2Y receptor-mediated biological activity.

A cell-free system for the study of P2Y receptors is also disclosed. The system can comprise a P2Y receptor; and a vesicle, and can further comprise a protein that is normally associated with the P2Y receptor in nature.

A method for producing a cell-free system for the assay of P2Y receptor-mediated activity is further disclosed. The method comprises purifying a P2Y receptor; purifying at least one protein that is normally associated with the P2Y receptor in nature; reconstituting the P2Y receptor into a vesicle; and reconstituting at least one protein that is normally associated with the P2Y receptor in nature into a vesicle to thereby produce a cell-free system.

Accordingly, it is an object of the present invention to provide an assay method and system for monitoring binding between a ligand and a P2Y receptor. The object is achieved in whole or in part by the present invention. An object of the invention having been stated hereinabove, other objects will become evident as the description proceeds when taken in connection with the accompanying Figures and Laboratory Examples as best described herein below.

### BRIEF DESCRIPTION OF THE DRAWINGS

FIG. 1 is a line graph depicting 2MeSADP-promoted steady state GTP hydrolysis by P2Y₁-R/G₁₁ in proteoliposomes. Purified human P2Y₁-R, Gα₁₁, and Gβ₁y₂ were reconstituted in phospholipid vesicles. GTP hydrolysis was quantitated at 30° C. in the presence of 100 nM RGS4 and in the absence of added vesicles (∘), in the presence of proteoliposomes (Δ), or in the presence (▴) of proteoliposomes plus 1 μM 2MeSADP.

FIGS. 2A and 2B depict agonist and antagonist activities quantitated with purified P2Y₁-R reconstituted in proteoliposomes with Gα₁₁. Purified P2y₁-R, Gα₁₁, and Gβ₁γ₂ were reconstituted in proteoliposomes.

FIG. 2A is a line graph depicting steady state GTP hydrolysis measured in proteoliposomes incubated in the absence (□) or presence (▴) of 100 nM RGS4 and the indicated concentrations of 2MeSADP.

FIG. 2B is a line graph depicting steady state GTP hydrolysis measured in proteoliposomes incubated with 100 nM RGS4, with the indicated concentrations of MRS2279, and with (▴) or without (Δ) 1 μM 2MeSADP.

FIGS. 3A and 3B depict the selectivity of coupling of P2Y₁-R to Gαq versus Gαo. Purified P2Y₁-R was reconstituted with Gβ1y2 and either purified Gαq or Gαo.

FIG. 3A is a bar graph depicting steady state GTP hydrolysis observed when P2Y₁-R/Gq proteoliposomes were incubated with 1 μM 2MeSADP and 100 nM RGS4 as indicated.

FIG. 3B is a bar graph depicting steady state GTPase hydrolysis observed when P2Y₁-R/Go proteoliposomes were incubated with 1 μM 2MeSADP and 100 nM RGS4 as indicated.

FIG. 4 is a line graph depicting promotion of 2MeSADP-stimulated GTPase activity by RGS2 and RGS4. Purified P2Y₁-R was reconstituted with Gαq and Gβ1y2. Steady state GTPase activity was measured in the presence of 1 μM 2MeSADP and the indicated concentrations of RGS2 (▪) or RGS4 (●).

FIG. 5 is a line graph depicting promotion of 2MeSADP-stimulated GTP hydrolysis by phospholipase C-β1. P2Y₁-R was reconstituted with Gαq and Gβ1y2. Steady state GTPase activity was measured in the absence (Δ) or presence (&Hslashed;) of 1 μM 2MeSADP and the indicated concentrations of PLC-β1. GTP hydrolysis in the presence of 100 nM RGS4 and in the absence (open bar) or presence (filled bar) of 1 μM 2MeSADP also was assessed.

### DETAILED DESCRIPTION OF THE INVENTION

The present invention pertains to the use of purified receptor protein or proteins for a rapid and sensitive assay of P2Y receptors. The assay is equally applicable to all of the cloned P2Y receptors, and preferred embodiments comprise purified P2Y₁, P2Y₂, P2Y₄, P2Y₆ and P2Y₁₁ receptors, with P2Y₁ and P2Y₂ receptors being most preferred. The present invention also provides a ligand binding assay for the P2Y receptors, and a preferred embodiment comprises a radioligand binding assay.

Prior to the disclosure of the present invention, it has not been possible to directly assess P2Y receptor ligand binding because expressed P2Y receptors in any tissue represent a very minor fraction of the total amount of nucleotide binding proteins. Therefore, binding of the relatively non-selective ligands that are available for the P2Y receptors occurs in much greater amount to other proteins. Thus, non-specific binding is very high, and obscures P2Y receptor binding. By developing methodology to purify functional P2Y-receptors to homogeneity in accordance with the present invention, a seminal advance that circumvents problems with non-receptor binding of ligands has been made.

A. Definitions

While the following terms are believed to have well defined meanings in the art, the following definitions are set forth to facilitate explanation of the invention.

As used herein, the term “labeled” means the attachment of a moiety, capable of detection by spectroscopic, radiologic or other methods, to a probe molecule.

As used herein, the term “nucleotide” refers to a phosphate ester of a nucleoside, and preferably, to 5′ triphosphate esters of the five major bases of DNA and RNA. The term “nucleotide” therefore includes ribonucleoside triphosphates (NTP's), e.g. ATP, CTP, UTP and GTP. The NTP's can be labeled with detectable label for use in the method of the present invention. The term “nucleotide” as used herein and in the claims is also meant to refer to nucleoside diphosphate molecules. The term “nucleoside diphosphate” includes ribonucleoside diphosphates (NDP's), e.g. ADP, CDP, UDP and GDP. Modified nucleotide bases (e.g. methylated bases) are also contemplated.

As used herein, the term “vesicle” means an enclosed and sealed bladder-like structure having an internal core and being capable of containing and supporting an integrated chemical entity. The term encompasses those structures commonly referred to as “liposomes”, “matrix vesicles”, “phospholipid vesicles” and similar structures known in the art.

As used herein, the term “candidate substance” means a substance that is believed to interact with another moiety, for example a given ligand that is believed to interact with a complete, or a fragment of, a P2Y receptor, and which can be subsequently evaluated for such an interaction. Representative candidate substances include xenobiotics such as drugs and other therapeutic agents, carcinogens and environmental pollutants, natural products and extracts, as well as endobiotics such as steroids, fatty acids and prostaglandins. Other examples of candidate substances that can be investigated by the assay method of the present invention include, but are not restricted to, agonists and antagonists for P2Y receptors, toxins and venoms, viral epitopes, hormones (e.g., opioid peptides, steroids, etc.), hormone receptors, peptides, enzymes, enzyme substrates, co-factors, lectins, sugars, synthetic or natural or antisense oligonucleotides or nucleic acids, oligosaccharides, proteins, and monoclonal antibodies.

As used herein, the term “protein normally associated with P2Y” means a protein that is normally associated with the P2Y receptor, as the receptor exists in the cell. Associated proteins and polypeptides can be those that permit the P2Y receptor to mediate its various biological activities. Associated proteins and polypeptides can also be those having roles that have not been clearly implicated in P2Y activity, yet are found in close spatial proximity to a P2Y receptor at a given point in time.

As used herein, the term “biological activity” means any observable effect resultant from the interaction between a P2Y receptor and a ligand. Representative, but non-limiting, examples of biological activity in the context of the present invention include hydrolysis of NTP molecules to NDP molecules, formation of NTP molecules from NDP molecules, modulation of intracellular calcium levels, modulation of phospholipase C activity, modulation of adenylate cyclase activity, translocation of RhoA to membranes, the formation of a network of stress fibers, phosphorylation of myosin light chains, cell differentiation, modulation of NTPase activity and shape change in platelets.

As used herein, the term “receptor-mediated activity” means any observable effect resulting directly from the binding of a ligand to a P2Y receptor, including the binding event itself. Receptor-mediated activity can be traced immediately to a P2Y binding event and is not an observed secondary, peripheral or phenotypic effect of the binding event.

As used herein, the term “modified” means an alteration from an entity's normally occurring state. An entity can be modified by removing discrete chemical units or by adding discrete chemical units. The term “modified” encompasses detectable labels as well as those entities added as aids in purification.

As used herein, the term “target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “transcription” means a cellular process involving the interaction of an RNA polymerase with a gene that directs the expression as RNA of the structural information present in the coding sequences of the gene. The process includes, but is not limited to, the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

As used herein, the term “expression” generally refers to the cellular processes by which a biologically active polypeptide is produced from RNA.

As used herein, the term “transcription factor” means a cytoplasmic or nuclear protein which binds to such gene, or binds to an RNA transcript of such gene, or binds to another protein which binds to such gene or such RNA transcript or another protein which in turn binds to such gene or such RNA transcript, so as to thereby modulate expression of the gene. Such modulation can additionally be achieved by other mechanisms; the essence of “transcription factor for a gene” is that the level of transcription of the gene is altered in some way.

As used herein, the term “hybridization” means the binding of a probe molecule, a molecule to which a detectable moiety has been bound, to a target sample.

As used herein, the term “detecting” means confirming the presence of a target entity by observing the occurrence of a detectable signal, such as a radiologic or spectroscopic signal that will appear exclusively in the presence of the target entity.

As used herein, the term “sequencing” means the determining the ordered linear sequence of nucleic acids or amino acids of a DNA or protein target sample, using conventional manual or automated laboratory techniques.

As used herein, the term “isolated” means oligonucleotides substantially free of other nucleic acids, proteins, lipids, carbohydrates or other materials with which they can be associated, such association being either in cellular material or in a synthesis medium. The term can also be applied to polypeptides, in which case the polypeptide will be substantially free of nucleic acids, carbohydrates, lipids and other undesired polypeptides.

As used herein, the term “substantially pure” means that the polynucleotide or polypeptide is substantially free of the sequences and molecules with which it is associated in its natural state, and those molecules used in the isolation procedure. The term “substantially pure” also encompasses purification of a polynucleotide or a polypeptide to near homogenity. The term “substantially free” means that the sample is at least 50%, preferably at least 70%, more preferably 80%, even more preferably 90%, and most preferably 99% free of the materials and compounds with which is it associated in nature.

As used herein, the term “primer” means a sequence comprising two or more deoxyribonucleotides or ribonucleotides, preferably more than three, and more preferably more than eight and most preferably at least about 20 nucleotides of an exonic or intronic region. Such oligonucleotides are preferably between ten and thirty bases in length.

As used herein, the term “promoter” includes what is referred to in the art as an upstream promoter region, a promoter region or a promoter of a generalized eukaryotic RNA Polymerase II transcription unit.

As used herein, the term “DNA segment” means a DNA molecule that has been isolated free of total genomic DNA of a particular species. Furthermore, a DNA segment encoding a P2Y receptor, yet is isolated away from, or purified free from, total genomic DNA of a source species, such as Homo sapiens. Included within the term “DNA segment” are DNA segments and smaller fragments of such segments, and also recombinant vectors, including, for example, plasmids, cosmids, phages, viruses, and the like.

As used herein, the phrase “enhancer-promoter” means a composite unit that contains both enhancer and promoter elements. An enhancer-promoter is operatively linked to a coding sequence that encodes at least one gene product.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

Following long-standing patent law convention, the terms “a” and “an” mean “one or more” when used in this application, including the claims.

B. Expression Vector Construction

Where a P2Y receptor gene itself is employed to express a P2Y receptor gene product, a convenient method of introduction will be through the use of a recombinant vector that incorporates the desired gene, together with its associated control sequences. In general, the preparation of recombinant vectors is well known to those of skill in the art and described in many references, such as, for example, Brown et al., Yeast 16(1):11-22 (2000) and Sambrook et al. (1992) Molecular Cloning: A Laboratory Manual (Cold Spring Harbor Laboratory Press, Cold Spring Harbor, N.Y.), specifically incorporated herein by reference.

Thus, a recombinant vector is provided in accordance with the present invention. The recombinant vector comprises a nucleic acid segment (e.g. a DNA segment) encoding a P2Y receptor, and is used for expressing a P2Y receptor. The recombinant vector can comprise a nucleic acid segment encoding any member of the P2Y receptor subfamily. The P2Y receptor can originate from any desired source, including but not limited to mammalian (e.g. human, rodent or other mammal), insect or other suitable species as would be apparent to one of ordinary skill in the art after review of the disclosure of the present invention presented herein. Representative P2Y receptors include those known to date, for example, the P2Y, receptor (Webb et al (1993) FEBS Lett. 324: 219-25; Schachter et al. (1996) Br. J. Pharmacol. 118: 167-73); the P2Y₂ receptor (Lustig et al. (1993) Proc. Natl. Acad. Sci. U S A 90: 5113-17); the P2Y₄ receptor (Communi et al. (1995) J. Biol. Chem. 270: 30849-52; Nguyen et al. (1995) J. Biol. Chem. 270: 30845-48); the P2Y₆ receptor (Chang et al. (1995) J. Biol. Chem. 270: 26152-58; Nicholas et al. (1996) Mol. Pharmacol. 50: 224-29) and the P2Y₁₁ receptor, which is a selective purinoreceptor and is dually coupled to both PLC and adenylate cyclase stimulation (Communi et al. (1999) Brit. J. Pharmacol. 128: 6 1199-206; Boeynaems et al. (2000) Trends Pharmacol. Sci. 21:1-3; WO 99/02675A1). Representative P2Y receptors, including sequence data, are also disclosed in U.S. Pat. No. 5,596,088; PCT Publication No. WO99/55901; U.S. Pat. No. 6,063,582; and PCT Publication No. WO97/19170, the entire contents of each of which are herein incorporated by reference. Other candidate receptors will likely be available in the future, and such receptors are encompassed by the present invention.

It is also envisioned that fusion proteins can be engineered in the present invention. Such a fusion protein can comprise a P2Y receptor and another protein, preferably a protein or polypeptide that normally associates with the P2Y receptor in nature. Candidates for fusion with a P2Y receptor include, but are not limited to, G_{q}α, G_{q}β, G_{q}γ, G_{12/13}α, G_{12/13}β, G_{12/13}γ, Gᵢα, Gᵢβ, Gᵢγ, Gₛα,Gₛβ, Gₛγ, Gβγ dimers, and combinations thereof. It is also envisioned that a fusion protein can comprise a P2Y receptor and a detectable protein or polypeptide, including but not limited to green fluorescent protein. Such a fusion protein can find application as a monitor of binding events, as a purification aid, and can have a role in detecting P2Y receptor-promoted biological activity. Any such fusion protein would be engineered using a vector design strategy as disclosed herein, as well as techniques and strategies known to those of skill in the art.

In vectors, it is understood that the DNA coding sequences to be expressed, in this case those encoding the P2Y receptor gene product, are positioned adjacent to and under the control of a promoter. It is understood in the art that to bring a coding sequence under the control of such a promoter, one generally positions the 5′ end of the transcription initiation site of the transcriptional reading frame of the gene product to be expressed between about 1 and about 50 nucleotides “downstream” of (i.e., 3′ of) the chosen promoter. One might also desire to incorporate into the transcriptional unit of the vector an appropriate polyadenylation site (e.g., 5′-AATAAA-3′), if one was not contained within the original inserted DNA. Typically, these poly A addition sites are placed about 30 to 2000 nucleotides “downstream” of the coding sequence at a position prior to transcription termination.

While use of the control sequences of the specific gene (i.e., the P2Y promoter for P2Y) will be preferred, there is no reason why other control sequences could not be employed, so long as they are compatible with the genotype of the cell being treated. Thus, one can mention other useful promoters by way of example, including, e.g., an SV40 early promoter, a long terminal repeat promoter from retrovirus, an actin promoter, a heat shock promoter, a metallothionein promoter, and the like.

As is known in the art, a promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes.

Another type of discrete transcription regulatory sequence element pertinent to the present invention is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites, as long as a promoter is present.

An enhancer-promoter used in a vector construct of the present invention can be any enhancer-promoter that drives expression in a cell to be transfected. By employing an enhancer-promoter with well-known properties, the level and pattern of gene product expression can be optimized.

For introduction of, for example, the P2Y gene, one can preferably employ a vector construct that will deliver the desired gene to a target cell. Delivery of the construct to a target cell can be achieved most preferably by introduction of the desired gene through the use of a viral vector to carry the P2Y sequence to efficiently infect the cells. These vectors will preferably be a baculoviral, an adenoviral, a retroviral, a vaccinia viral vector, an adeno-associated virus, or other suitable vector as would be apparent to one of ordinary skill in the art after review of the disclosure of the present invention presented herein. These vectors are preferred because they have been successfully used to deliver desired sequences to cells and tend to have a high infection efficiency. Thus, in one embodiment, a recombinant vector of the present invention further comprises: (a) a sequence of genomic viral DNA showing affinity for a host cell and possessing the ability to infect said host cell; (b) a nucleic acid sequence encoding a P2Y receptor operatively linked to the sequence of genomic viral DNA, wherein the operatively-linked P2Y receptor is expressed in said host cell following infection of the cell; and (c) a selectable marker.

Commonly used viral promoters for expression vectors are derived from polyoma, cytomegalovirus, Adenovirus 2, and Simian Virus 40 (SV40). The early and late promoters of SV40 virus are particularly useful because both are obtained easily from the virus as a fragment that also contains the SV40 viral origin of replication. Smaller or larger SV40 fragments can also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

The origin of replication can be provided either by construction of the vector to include an exogenous origin, such as can be derived from SV40 or other viral (e.g., Polyoma, Adeno, VSV, BPV) source, or can be provided by the host cell chromosomal replication mechanism. If the vector is integrated into the host cell chromosome, the latter is often sufficient.

Where a P2Y gene itself is employed it will be most convenient to simply use a wild type P2Y gene directly. It is envisioned, however, that certain regions of a P2Y gene can be employed exclusively without employing an entire wild type P2Y gene. It is proposed that it will ultimately be preferable to employ the smallest region needed to modulate cell signaling so that one is not introducing unnecessary DNA into the system. Techniques well known to those of skill in the art, such as the use of restriction enzymes, will allow for the generation of small regions of a P2Y gene. The ability of these regions to modulate cell signaling can be determined in accordance with the assay method of the present invention.

An expression vector of the present invention can also comprise nucleic acid segments that encode other proteins or peptides having desired functions, such as for purification or immunodetection purposes. For example, the expressed receptors can further comprise hexahistidine tags to assist in purification and/or FLAG®-epitope tags (Immunex Corporation, Seattle, Wash.) for immuno-identification and to further aid in purification.

A method of preparing a P2Y receptor is also provided in accordance with the present invention. The method comprises transfecting a cell with a recombinant vector comprising a P2Y receptor-encoding nucleic acid segment under conditions suitable for the expression of the receptor, to thereby produce a P2Y receptor. The cell can be a prokaryotic cell or a eukaryotic cell. Optionally, a P2Y receptor can be expressed in a unique human cell line, such as 1321N1 human astrocytoma cells. This can be accomplished using standard cloning techniques described herein and in the art.

In a preferred embodiment of the present invention, baculoviral vectors are engineered for high level expression of P2Y receptors in insect cells, more preferably Sf9 insect cells, and have a mammalian or insect signal sequence preceding the N-terminal epitope tag. In general, the use of baculovirus expression systems is well known to those of skill in the art. Protocols are available in conjunction with commercially available baculovirus kits for expression of engineered P2Y.

C. Cell-free Model System

In another aspect of the present invention, a cell-free system for the study of P2Y receptors is provided. The cell free system of the present invention makes it possible for researchers to study P2Y receptors and P2Y receptor-mediated activity in vitro.

In a preferred embodiment, a cell-free system of the present invention for the study of P2Y receptors comprises a P2Y receptor; a protein that is normally associated with the P2Y receptor in nature; and a vesicle. Representative vesicles and techniques for preparing the same are described below.

Representative P2Y receptors include but are not limited to the P2Y₁ receptor, the P2Y₂ receptor, the P2Y₄ receptor, the P2Y₆ receptor and the P2Y₁₁ receptor. Preferably, the P2Y receptor is a P2Y₁ or P2Y₂ receptor. More preferably, the P2Y receptor in the cell-free system is substantially pure.

The cell-free system of the present invention can also comprise a protein that normally associates with the P2Y receptor in nature. This is a distinct advantage because it allows researchers to closely model the native in situ environment of a P2Y receptor. The advantage in such a model is that it makes it possible to monitor activities related to, but distinct from, a P2Y receptor-ligand binding event. In the cell-free system of the present invention, the proteins that are normally associated with a P2Y receptor in nature can be G proteins. Representative G proteins include but are not limited to G_{q}α, G_{q}β, G_{q}γ, G₁₁α, G_{12/13}α, G_{12/13}β, G_{12/13}γ, Gᵢα, Gᵢβ, Gᵢγ, Gₛα, Gₛβ, Gₛγ, Gα₁₄ and Gα₁₆, various Gβγ dimers, and combinations thereof. Preferably, the associated protein is also substantially pure.

Optionally, the system further comprises a ligand for the P2Y receptor or for the protein that normally associates with the P2Y receptor in nature. Representative ligands include NTP, NDP, modified forms thereof, and combinations thereof. For example, UTPγS and ATPγS can be employed as a high affinity ligand for the P2Y₁ and P2Y₂ receptor respectively, both of which can be synthesized with ³⁵S and employed as a radioligand.

Representative ligands also include GTPase activating proteins (GAPs), such as RGS (regulator of G protein signaling) proteins. RGS proteins are potent GAPs, accelerating the slow intrinsic rate of GTP hydrolysis by G(x proteins and thus converting them to their inactive GDP-bound forms. Representative RGS proteins include but are not limited to RGS1, RGS2, RGS4 and RGS16. Indeed, any of the over 20 RGS proteins expressed in mammals can be employed in a system of the present invention. See Zeng et al., (1998) J. Biol. Chem. 273(52):34687-34690; Xu et al., (1999) J. Biol. Chem. 274(6):3549-3556; and Mukhopadhyay et al., (1999) Proc. Natl. Acad. Sci. USA 96:9539-9544.

Representative ligands also include art-recognized agonists and antagonists of a P2Y receptor. As disclosed in the Laboratory Examples presented below, a bisphosphate antagonist of the P2Y₁ receptor can be radiolabeled and used as a radioligand.

In another embodiment, a cell-free system of the present invention for the study of P2Y receptors comprises a P2Y receptor and a vesicle. Receptor binding events can be monitored using this embodiment of the cell-free system, and can be used to screen for modulators as described herein.

C.1. Labeling of System Components

Receptor binding events in the cell-free system of the present invention can be conveniently monitored by labeling a system component. Preferably, a ligand for a P2Y receptor is labeled. More preferably, the labeled ligand is an NTP, an NDP, or a combination thereof. Most preferably, the labeled ligand is radiolabeled for easy detection, although other labels are envisioned and will be apparent to one of skill in the art. Representative radioisotopes for labeling include but are not limited to ³H, ³²P, ³⁵S, ¹⁴C and ¹²⁵I.

Fluorescent compounds can be used to label a P2Y receptor, a protein normally associated with a P2Y receptor and/or a ligand (e.g. a nucleotide) in accordance with the present invention. Representative fluorescent labeling compounds include near-infrared fluorescent dyes and also include dinitrophenyl, fluorescein and derivatives thereof (such as fluorescein isothiocyanate), rhodamine, derivatives of rhodamine (such as methylrhodamine and tetramethylrhodamine), phycoerythrin, phycocyanin, allophycocyanin, o-phthaldehyde and fluorescamine. Representative fluorescent dyes include Texas red, Rhodamine green, Oregon green, Cascade blue, phycoerythrin, CY3, CY5, CY2, CY7, coumarin, infrared 40, MR 200, and IRD 40. Representative chemiluminescent labeling compounds are luminol, isoluminol, theromatic acridinium ester, imidazole, acridinium salt and oxalate ester, while representative bioluminescent compounds for purposes of labeling are luciferin, luciferase and aequorin. Green fluorescent protein (GFP) will also be of use as a fluorescent marker. All of the compounds are available from commercial sources, such as Cienca, Inc. of East Hartford, Conn.; Molecular Probes, Inc., Eugene, Oreg.; and Sigma Chemical Company, St. Louis, Mo.

Fluorescent labeled nucleotides are also commercially available from Boehringer Mannheim, Indianapolis, Ind.; Pharmacia Biosystems Aktiebolaget, Uppsala, Sweden; NEN-Dupont, Wilmington, Del.; and Molecular Probes, Inc., Eugene, Oreg.

Additionally, in accordance with the present invention, two system components each can be labeled with an energy emitting moiety (i.e. an energy contributing donor moiety and an energy receiving acceptor moiety) so that a detectable signal can be generated from resonant interaction between the two energy emitting moieties. For example, a P2Y receptor can be labeled with the donor moiety while a protein normally associated with a P2Y receptor can be labeled with the acceptor moiety, and vice versa. In either case, an appropriate spatial relationship for resonance energy transfer (RET) between the energy-emitting moiety is provided. RET is described in U.S. Pat. Nos. 4,058,732 and 4,374,120 and in Sineav et al., Bioconjugate Chem. 11:352-362 (2000), incorporated by reference herein.

The term “energy-emitting moiety” is believed to be well understood by one of skill in the art and is meant to refer to any moiety, whether an atom, molecule, complex or other moiety, that emits energy in response to a stimulus. The methods of the present invention are contemplated to be useful for any combinations of energy-emitting moiety so long as the emitted energy from one moiety is sufficiently intense so as to produce as an energy emission from the other moiety in accordance with the present invention. For example, energy transfer can occur when the emission spectrum of the donor overlaps the absorption spectrum of the acceptor. Thus, acceptor and donor moieties can be chosen and paired together based on these characteristics. Also, the donor and the acceptor must be within a certain distance, i.e. preferably within the same complex, from each other.

Preferred “energy-emitting moieties” comprise luminescent or light emitting molecules, such as fluorescent, phosphorescent, and chemiluminescent molecules, which emit light when excited by excitation light. Preferred donor/acceptor combinations that can be used in the present inventive method are fluorescent donors with fluorescent or phosphorescent acceptors, or phosphorescent donors with phosphorescent or fluorescent acceptors.

C.2. Vesicle Preparation

As used herein, the term “vesicle” means an enclosed and sealed bladder-like structure having an internal core and being capable of containing and supporting an integrated chemical entity. The term encompasses those structures commonly referred to as “liposomes”, “matrix vesicles” and “phospholipid vesicles”.

Vesicles are spherical structures having a lipid layer surrounding a central space. The present invention is particularly concerned with unilammellar and multilamellar vesicles which have, respectively, a single lipid bilayer or multiple lipid bilayers surrounding an aqueous core. Vesicles spontaneously form upon dispersion of lipids, particularly phospholipids, in aqueous media and the liposomal structure of the agents of the invention can be produced by conventional techniques. Such conventional techniques are referred to in WO92/21017 (Unger), and Papahadjopolous (1979) Ann Rep. Med. Chem. 14: 250-60. Such techniques include reverse evaporation, freeze-thaw, detergent dialysis, homogenization, sonication, microemulsification and spontaneous formation upon hydration of a dry lipid film. Multi-lamellar vesicles can be used according to the invention or can be converted to vesicles with lower lamellarity, or to unilamellar vesicles, by known methods. Unilamellar vesicles can also be prepared directly.

Vesicle preparations are typically heterogeneous in size and the vesicles used according to the invention can be sized to the desired diameter by known techniques, e.g. extrusion, freeze-thaw, mechanical fragmentation, homogenization and sonication. The vesicles used according to the invention are advantageously 20-5000 nm diameter, unilamellar or multi-lamellar. The vesicles can be lyophilized to increase shelf life and lyophilized vesicles can be reconstituted by vigorous shaking with aqueous buffer prior to use. Formulations can include agents that serve to stabilize the vesicle material for the lyophilization procedure. Vesicles smaller than 200 nm can be sterilized after formulation by filtration.

The lipids used as the lipid bilayer-forming, or vesicle-forming, molecules are typically phospholipids or hydrogenated phospholipids, such as natural or synthetic phosphatidylcholines (lecithins) (PC), phosphatidylethanolamines (PE), lysolecithins, lysophosphatidylethanolamines, phosphatidylserines (PS), phosphatidylglycerols (PG), phosphatidylinositol (PI), sphingomyelins, cardiolipin, phosphatidic acids (PA), fatty acids, gangliosides, glucolipids, glycolipids, mono-, di or triglycerides, ceramides or cerebrosides, e.g. vesicle membrane forming compounds such as are described in WO92/21017.

Bilayer- or vesicle-forming lipids can also comprise polymerizable lipids, e.g. methacrylate lipids, thiol and disulphide lipids, dienoate lipids, styryl lipids and diacetylanic lipids as described by Johnston ((1983) Liposome Technology Vol. I, Gregoriades Ed., pages 123-29), Singh ((1993) Phospholipid Handbook, Cevc Ed., Dekker, pages 233-91) and references therein. The use of polymerizable lipids in the formation of the vesicles provides one route for increasing liposome stability.

The lipids forming the lipid bilayer or vesicle can also be cationic lipids, which have a lipophilic moiety, such as a sterol, an acyl or diacyl chain, and where the lipid has an overall net positive charge. Preferably, the head group of the lipid carries the positive charge. Exemplary cationic lipids include 1,2-dioleyloxy-3-(trimethylamino) propane (DOTAP); N-[1-(2,3,-ditetradecyloxy) propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy)propyl]-N,N,N-trimethylammonium chloride (DOTMA); 3-[N-(N′,N′-dimethylaminoethane)carbamoly]cholesterol (DC-Chol); and dimethyidioctadecylammonium (DDAB).

The cationic vesicle- or lipid bilayer-forming lipid can also be a neutral lipid, such as dioleoylphosphatidyl ethanolamine (DOPE), cholesterol-containing DOPC, or an amphipathic lipid, such as a phospholipid, derivatized with a cationic lipid, such as polylysine or other polyamine lipids. For example, the neutral lipid (DOPE) can be derivatized with polylysine to form a cationic lipid.

The lipid bilayer or vesicle membrane can also have steroids and other compounds incorporated into it, e.g. to affect the biodistribution of the liposome. Suitable steroids include for example cholesterol, cholesterol derivatives, cholestane, cholic acid, and bile acids, but particularly cholesterol. The inclusion of steroids serves to modify the fluidity of the liposome membrane and the inclusion of cholesterol results in a more rigid and less permeable bilayer.

Representative starting materials and method for the preparation of vesicles are also disclosed in U.S. Pat. Nos. 6,048,546; 6,045,821; 6,045,822; and 6,043,094. The entire contents of each of these U.S. patents are incorporated by reference herein.

C.3. Preparation Methods

The cell-free system of the present invention is produced by a method comprising: purifying a P2Y receptor; and reconstituting the P2Y receptor into a vesicle. The method can further comprise purifying at least one protein that is normally associated with the P2Y receptor in nature; and reconstituting at least one protein that is normally associated with the P2Y receptor in nature into the vesicle to thereby produce a cell-free system.

A typical purification scheme generally begins by expressing P2Y receptors, rupturing the cells expressing P2Y receptors and subsequently isolating, as nearly as possible, the expressed P2Y receptor from cellular debris and entities naturally associating with the P2Y receptor in the cell. This is accomplished by a combination of centrifugation and chromatography. Representative purification techniques are disclosed by Biddlecome et al., J. Biol. Chem. 271 (14):7999-8007; by Brown et al., Yeast 16(1):11-22 (2000); and by Sambrook et al. (1992) Molecular Cloning: A Laboratory Manual (Cold Spring Harbor Laboratory Press, Cold Spring Harbor, N.Y.).

The purification progresses by taking advantage of hexahistidine tags, which are engineered to append to the C-terminal or N-terminal end of the polypeptide. The purification can alternatively be assisted by the presence of an engineered epitope. Optionally, the epitope is engineered to append to a terminal end of the polypeptide.

The P2Y receptor is easily purified by either passing the partly-purified sample over a nickel-NTA column, which will bind the hexahistidine tag, or by immunological methods that take advantage of the engineered epitope. The polypeptide can subsequently be eluted from the nickel column or the immunological purification aid. Standard protein purification methodology is employed in conjunction with and throughout the above general scheme. The purification scheme results in a suspension in which P2Y receptors are purified to near homogeneity, i.e. are substantially pure, as defined herein. As an alternative aid in purification, a P2Y receptor can be engineered to expresses a FLAG® epitope at either the N-terminal or C-terminal end of a P2Y receptor protein. In this purification scheme, the P2Y receptor is purified by binding the receptor to a detectable anti-FLAG® antibody. The immunocomplex can subsequently be isolated.

A protein, or proteins, that is (or are) normally associated with the P2Y receptor in nature can be purified using methodology disclosed by Cosawa and Gilman, (1995) J. Biol. Chem. 270: 1734-41. The effectiveness of several detergents was also compared, and excellent solubilization, purification, and functional reconstitution utilizing digitonin was observed. Other detergents evaluated include dodecylmaltoside and CHAPS. These detergents were effective, but are less preferred than digitonin.

Throughout the purification of a P2Y receptor, e.g. the P2Y₂ receptor, 100 mM phosphate is preferably maintained. Following detergent solublization, protease inhibitors are preferably included to maintain the integrity of the receptor. Representative protease inhibitors include but are not limited to TPCK, PMSF, Leupeptin, Pepstatin A, aprotinin and ABSF.

A P2Y receptor that is reconstituted into the cell-free system of the present invention, as well as the protein, or proteins, that are normally associated with the P2Y receptor in nature, can be expressed in an expression system. In a preferred embodiment, a P2Y receptor, as well as the protein, or proteins, that are normally associated with the P2Y receptor in nature, are expressed in a baculovirus expression system in accordance with techniques disclosed hereinabove.

In a preferred embodiment, the purified P2Y receptor is reconstituted in a vesicle alone or with a purified protein, or proteins, that is (or are) normally associated with the P2Y receptor in nature. Representative reconstitution techniques are disclosed by Biddlecome et al., J. Biol. Chem. 271(14):7999-8007; by Brown et al., Yeast 16(1):11-22 (2000); and by Sambrook et al. (1992) Molecular Cloning: A Laboratory Manual (Cold Spring Harbor Laboratory Press, Cold Spring Harbor, N.Y.). Proteins are kept separate from each other prior to reconstitution in vesicles. Ligands are also preferably kept separate from other system components until an assay method of the present invention is to be executed.

As a preferred example, reconstitution of a P2Y receptor in a vesicle can be accomplished as follows. In a glass tube, the following lipid solutions, which are made by solubilizing the lipids in chloroform, are combined: 11 μl PE (10 mg/ml), 7 μl PS (10 mg/ml), 4 μl cholesteryl hemisuccinate (2 mM). The mixture is dried under a stream of N₂ or argon to prevent oxidation. The lipids are then dissolved in a buffer containing deoxycholate at 0.4% and sonicated to ensure complete solubilization. Proteins are added sequentially: 50 pmol of the appropriate G protein α subunit, 150 pmol Gβγ and 15 pmol of P2Y receptor. The mixture is passed over a sizing column to isolate the vesicles from the single components and the detergent. Isolated vesicles can then used for the GTP hydrolysis assays for which they can be diluted depending on their quality.

D. Screening Assays

In yet another aspect, the present invention provides a method of screening substances for their ability to affect or modulate the biological activity of P2Y receptor-promoted activity. The present invention also provides a process of screening substances for their ability to affect or modulate P2Y receptor-mediated biological activity to thereby affect or modulate the biological activity of other downstream proteins. A candidate substance is a substance that potentially can promote (i.e. agonize) or inhibit (i.e. antagonize) P2Y receptor-mediated biological activity by binding, or other intramolecular interaction, with the P2Y receptor itself. The terms “modulate” and “modulator” are thus used herein to encompass both promotion and inhibition of a P2Y receptor-mediated biological activity.

P2Y receptor-promoted biological activity can comprise NTP binding activity, cell signaling activity or other biological activity in accordance with the present invention. The P2Y receptor-promoted biological activity also includes but is not limited to hydrolysis of NTP molecules to NDP molecules, promotion of binding of NTP molecules such as GTPγS, modulation of intracellular calcium levels, modulation of phospholipase C activity, modulation of adenylate cyclase activity, translocation of RhoA to membranes, formation of a network of stress fibers, phosphorylation of myosin light chains, cell differentiation modulation of NTPase activity, shape change in platelets, or any combination thereof.

In one embodiment, a method of screening candidate substances for an ability to modulate P2Y receptor-promoted biological activity comprises: (a) establishing a test sample comprising a substantially pure P2Y receptor; (b) contacting the test sample with a candidate substance; and (c) measuring an interaction, effect, or combination thereof, of the candidate substance on the test sample to thereby determine the ability of the candidate substance to modulate P2Y receptor-promoted biological activity.

Another representative method of screening candidate substances for their ability to modulate P2Y receptor-promoted biological activity comprises: (a) establishing replicate test and control samples that comprise a substantially pure biologically active P2Y receptor polypeptide; (b) administering a candidate substance to test sample but not the control sample; (c) measuring the activity of P2Y receptor-promoted biological activity in the test and the control samples; and (d) determining that the candidate substance modulates P2Y receptor-promoted biological activity if the level of P2Y receptor-promoted activity measured for the test sample is greater or less than the level of P2Y receptor-promoted biological activity measured for the control sample.

In another embodiment, an assay method of the present invention comprises establishing a control system comprising a P2Y receptor and a ligand, wherein the P2Y is capable of binding to the ligand; establishing a test system comprising a P2Y receptor, a ligand, and a candidate compound; measuring the binding affinity of a P2Y receptor and a ligand in the control and the test systems; and determining that the candidate compound modulates P2Y receptor-promoted activity in a cell-free system if the binding affinity measured for the test system is less than or greater than the binding affinity measured for the control system.

Preferably, any embodiment of the method of the present invention is carried out using a cell-free system of the present invention. Thus, the test and control samples can further comprise a vesicle comprising a P2Y receptor and a protein that normally interacts with a P2Y receptor in nature. Representative P2Y receptors include but are not limited to the P2Y₁ receptor, the P2Y₂ receptor, the P2Y₄ receptor, the P2Y₆ receptor and the P2Y₁₁ receptor. Preferably, the P2Y receptor is a P2Y₁ or P2Y₂ receptor. More preferably, the P2Y receptor in the cell-free system is substantially pure.

Preferably, a protein that normally interacts with a P2Y receptor in nature is a G protein. More preferably, the protein that normally interacts with a P2Y receptor in nature is selected from the group including but not limited to G_{q}α, G_{q}β, G_{q}γ, G₁₁α, G_{12/13}α, G_{12/13}β, G_{12/13}γ, Gᵢα, Gᵢβ, Gᵢγ, Gₛα, Gₛ,β,Gₛγ, Gα₁₄, Gα₁₆, Gβγ dimers, and combinations thereof. Even more preferably, the protein that normally interacts with a P2Y receptor in nature is substantially pure.

Representative ligands include GTPase activating proteins (GAPs), such as RGS (regulator of G protein signaling) proteins. RGS proteins are potent GAPs, accelerating the slow intrinsic rate of GTP hydrolysis by GU proteins and thus converting them to their inactive GDP-bound forms. Representative RGS proteins include but are not limited to RGS1, RGS2, RGS4 and RGS16. Indeed, any of the over 20 RGS proteins expressed in mammals can be employed in a method of the present invention. See Zeng et al., J. Biol. Chem. 273(52):34687-34690 (Dec. 25, 1998); Xu et al., J. Biol. Chem. 274(6):3549-3556 (Feb. 5, 1999); and Mukhopadhyay et al., Proc. Natl. Acad. Sci. USA 96:9539-9544 (August 1999).

Representative ligands also include art-recognized agonists and antagonists of a P2Y receptor. Representative agonists and antagonists are disclosed in the Laboratory Examples presented below.

Receptor binding events in an assay method of the present invention can be conveniently monitored by labeling a system component. Preferably, a ligand for a P2Y receptor is labeled. More preferably, the labeled ligand is an NTP, an NDP, a high affinity receptor antagonist or a combination thereof. Most preferably, the labeled ligand is radiolabeled for easy detection, although other labels are envisioned and will be apparent to one of skill in the art. Representative radioisotopes for labeling include but are not limited to ³H, ³²P, ³⁵S, ¹⁴C and ¹²⁵I.

Fluorescent compounds can be used to label a P2Y receptor, a protein normally associated with a P2Y receptor and/or a ligand (e.g. a nucleotide) in accordance with the present invention. Representative fluorescent labeling compounds include near-infrared fluorescent dyes and also include dinitrophenyl, fluorescein and derivatives thereof (such as fluorescein isothiocyanate), rhodamine, derivatives of rhodamine (such as methylrhodamine and tetramethylrhodamine), phycoerythrin, phycocyanin, allophycocyanin, o-phthaldehyde and fluorescamine. Representative fluorescent dyes include Texas red, Rhodamine green, Oregon green, Cascade blue, phycoerythrin, CY3, CY5, CY2, CY7, coumarin, infrared 40, MR 200, and IRD 40. Representative chemiluminescent labeling compounds are luminol, isoluminol, theromatic acridinium ester, imidazole, acridinium salt and oxalate ester, while representative bioluminescent compounds for purposes of labeling are luciferin, luciferase and aequorin. All of the compounds are available from commercial sources, such as Cienca, Inc. of East Hartford, Conn.; Molecular Probes, Inc., Eugene, Ore.; and Sigma Chemical Company, St. Louis, Mo.

Fluorescent labeled nucleotides are also commercially available from Boehringer Mannheim, Indianapolis, Ind.; Pharmacia Biosystems Aktiebolaget, Uppsala, Sweden; NEN-Dupont, Wilmington, Del.; and Molecular Probes, Inc., Eugene, Oreg.

A presence or an amount of modulation of P2Y receptor-promoted activity in a test sample or in a control sample can be assessed in any suitable manner, such as for example, through the detection of a ligand. Preferably, the ligand is detectably labeled with a detectable moiety as described above. Alternatively, electromagnetic measurement techniques can be employed. By way of additional example, binding affinity can be assessed by comparing an amount of bound ligand in an experiment to the amount of unbound ligand in the experiment. In this case it is also preferable that the ligand be detectably labeled. Bound and unbound labeled ligands can be separated by contacting the reaction mixture with a separation matrix. Any suitable separation matrix as would be apparent to one of ordinary skill in the art after review the present disclosure is envisioned.

Additionally, in accordance with the present invention, a detectable signal can be generated from resonant interaction between two energy emitting moieties: an energy contributing donor moiety and an energy receiving acceptor moiety. For example, a P2Y receptor can be labeled with the donor moiety while a protein normally associated with a P2Y receptor can be labeled with the acceptor moiety, and vice versa. In either case, an appropriate spatial relationship for resonance energy transfer (RET) between the energy-emitting moiety is provided through the binding of the proteins in the presence of, for example, a candidate compound that interacts with the P2Y receptor to promote such binding. RET is described in U.S. Pat. Nos. 4,058,732 and 4,374,120, incorporated by reference herein.

D.1. Steady State Assay for P2Y Receptor-promoted Activity

In a preferred embodiment, a highly sensitive assay that measures steady state GTPase activity is provided. Importantly, the availability of purified P2Y receptors allows the direct assay of P2Y receptor binding and P2Y receptor-promoted activity using a ligand (e.g. a radioligand). Since binding to contaminating proteins, e.g., ATPases, is not a problem, labeled ADP can be utilized to measure, for example, P2Y₁ receptor binding, and labeled UTP to measure, for example, P2Y₂ receptor binding.

In a steady state embodiment of the present invention, vesicles are combined with agonists, GAP proteins γ-[³²P] labeled GTP, all in an appropriate buffer system. The assay is then incubated at between 20° C. and 30° C. for various times. P2Y receptor-promoted activity is measured as released ³²P, which is separated from [γ-³²P]GTP after the assay is halted by transfer to ice and addition of ice cold 5% slurry or activated charcoal in 20 mM phosphoric acid. See Biddlecome et al., J. Biol. Chem. 271: 7999-8007.

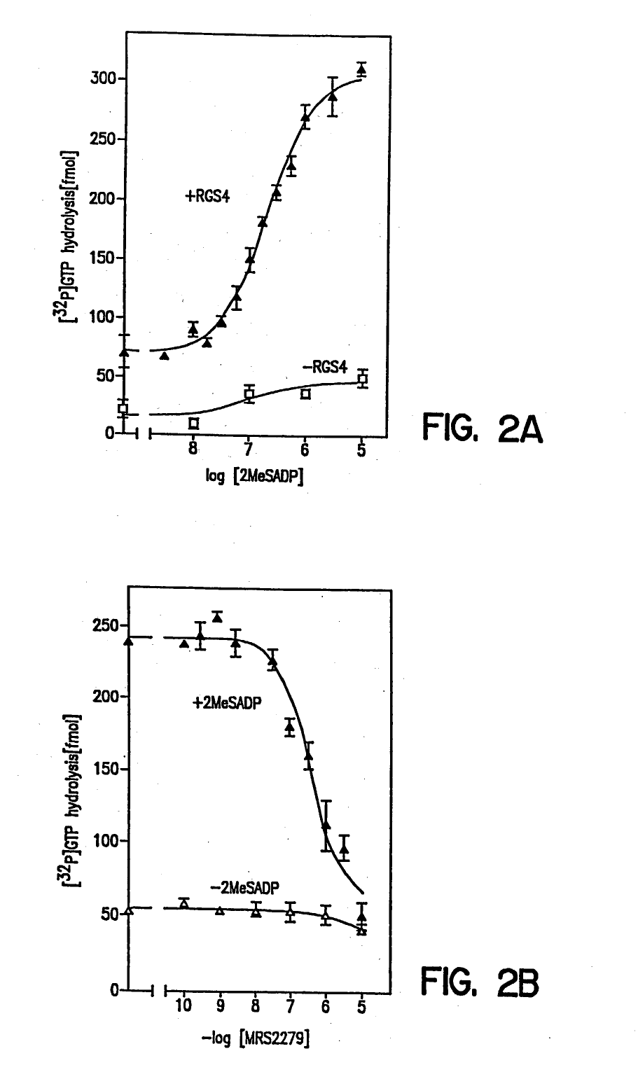

Gαq binds GDP with high affinity, and therefore, the basal GTPase concentration is very low compared to that in the presence of a P2Y receptor (e.g. P2Y₁ receptor) agonist (e.g. 2MeSADP) and an RGS protein (e.g. RGS4), which together stimulate GTPase activity by up to 100-fold. See FIG. 2A. Antagonists are identified by their ability to inhibit activity observed in the presence of an EC₇₀ concentration, approximately 50 nM, of a P2Y receptor (e.g. P2Y₁ receptor) agonist (e.g. 2MeSADP). See FIG. 2B.

D.2. Agonist and Antagonist Assays

In an alternative assay envisioned in accordance with the present invention, agonist-promoted [³⁵S]GTPγS binding is measured, instead of steady state GTPase activity.

The present invention also provides a bisphosphate antagonist of the P2Y₁ receptor as a ligand, which can be radiolabeled and used as a radioligand. The present invention also discloses UTPγS as a high affinity ligand for the P2Y₂ receptor, which can be synthesized with ³⁵S and employed as a radioligand. Standard means of separating receptor bound and free radioligand can be applied, and since non-receptor proteins have been eliminated in a preferred purification scheme, the signal to noise ratio of the assay is exceptionally high.

D.3. Rapid, High-Throughput Assay System

The present invention permits, for the first time, the use of a rapid, high-throughput system of assaying P2Y receptor binding and P2Y receptor-promoted activity. The cell-free system of the present invention eliminates contaminating protein and, therefore, non-specific binding. The elimination of contaminants normally present in cells greatly enhances the signal to noise ratio of the assay. Thus, due to the low degree of background signal, even weak binding events and low-level activities can be accurately detected and quantified.

A technique for drug screening which can be used in conjunction with the present invention provides for high throughput screening of compounds having suitable binding affinity to the protein of interest, as described in published PCT application WO 84/03564, herein incorporated by reference. In this method, as applied to the P2Y receptor polypeptide, large numbers of different small test compounds are synthesized, either in a solution or on a solid substrate, such as plastic pins or some other surface. The test compounds are reacted with the purified P2Y receptor in a cell free system of the present invention. A cell free system of the present invention can be loaded in a multi-well plate, such as a 96-well or 384-well plate. A cell free system of the present invention can also be coated directly onto plates for use, such as in a lipid bilayer (encompassed by the term “vesicle” as used herein), in the aforementioned drug screening techniques. An interaction between a candidate substance and a P2Y receptor polypeptide is then detected as disclosed herein and as are known in the art for screening of multiple samples in a single effort.

Robotic systems that are suitable for use in the methods of the present invention are commercially available from Beckman Coulter, Inc. of Fullerton, Calif. and are sold under the trademark SAGIAN™ and under the registered trademark BIOMEK® 2000. These systems are preferred for use in the transfer of candidate substances from 96-well and 384-well source plates a similar destination plate. A MULTIMEK™ 96 automated 96-channel pipettor (also available from Beckman Coulter, Inc. of Fullerton, Calif.) can be used in the transfer of candidate compounds between 96-well and 384-well source and destination plates.

D.4. Screening of P2Y Receptor-promoted Biological Activity

Any member of the P2Y receptor family can serve as a standard in a screening assay for biological activity mediated by the receptor binding event, in accordance with the present invention. For example, the P2Y₁ receptor promotes phospholipase C-catalyzed generation of inositol phosphates and subsequent mobilization of intracellular calcium. The mobilization of intracellular calcium is a common and important mechanism that regulates the activity of biological molecules in vivo. The P2Y₁ receptor has been determined to promote mobilization of intracellular calcium and therefore can be used as a standard or control in an assay to determine the calcium mobilization activity of another member of the P2Y receptor family.

### LABORATORY EXAMPLES

The following Laboratory Examples have been included to illustrate preferred modes of the invention. Certain aspects of the following Laboratory Examples are described in terms of techniques and procedures found or provided by the present inventors to work well in the practice of the invention. These Laboratory Examples are exemplified through the use of standard laboratory practices of the inventors. In light of the present disclosure and the general level of skill in the art, those of skill will appreciate that the following Laboratory Examples are intended to be exemplary only and that numerous changes, modifications and alterations can be employed without departing from the spirit and scope of the invention.

### Laboratory Example 1

The P2Y₁ receptor was expressed, purified and reconstituted into vesicles as described herein. GTP hydrolysis was measured by incubation of vesicles with 2 μM [γ³²P]GTP and quantitation of released [³²P]Pᵢ. The basal rate of GTP hydrolysis by Gα11 is low, and guanine nucleotide exchange is the rate-limiting step in the GTP hydrolytic cycle. In the presence of agonist, the rate-limiting step becomes GTP hydrolysis, and therefore the GTPase-stimulating protein, RGS4, was included in most experiments.

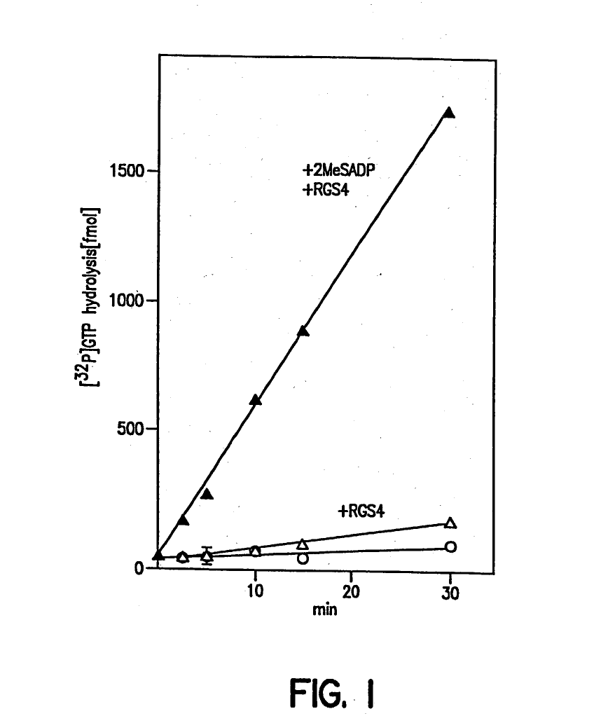

As depicted in FIG. 1, addition of 2-Methylthioadenosine diphosphate (2MeSADP) to vesicles reconstituted with the P2Y₁ receptor, Gα11, and Gβ1γ2 resulted in a marked increase in the hydrolysis of GTP in the presence of RGS4. GTP hydrolysis was linear for at least 45 minutes under these conditions and also was linearly dependent on the amount of vesicles in the assay.

The EC₅₀ of 2MeSADP (220 nM; FIG. 2A) was similar to that previously observed in inositol phosphate and Ca²⁺ measurements with the recombinant receptor expressed in 1321N1 human astrocytoma cells. MRS2279, a compound that was previously developed as an antagonist of the P2Y₁ receptor (Boyer et al. (1998) Brit. J. Pharmacol. 124: 1-3), also antagonized 2MeSADP-promoted GTPase activity with an IC₅₀ (FIG. 2B) similar to that observed in intact cell assays.

The availability of purified P2Y₁ receptor also provides a system to assess directly the activity of other molecules at this receptor. For example, disagreement exists in the art over the agonist versus antagonist nature of ATP with respect to P2Y receptors, and data obtained using the systems and methods of the present invention suggest that ATP is a pure antagonist of this P2Y receptor in the absence of receptor reserve.

### Laboratory Example 2

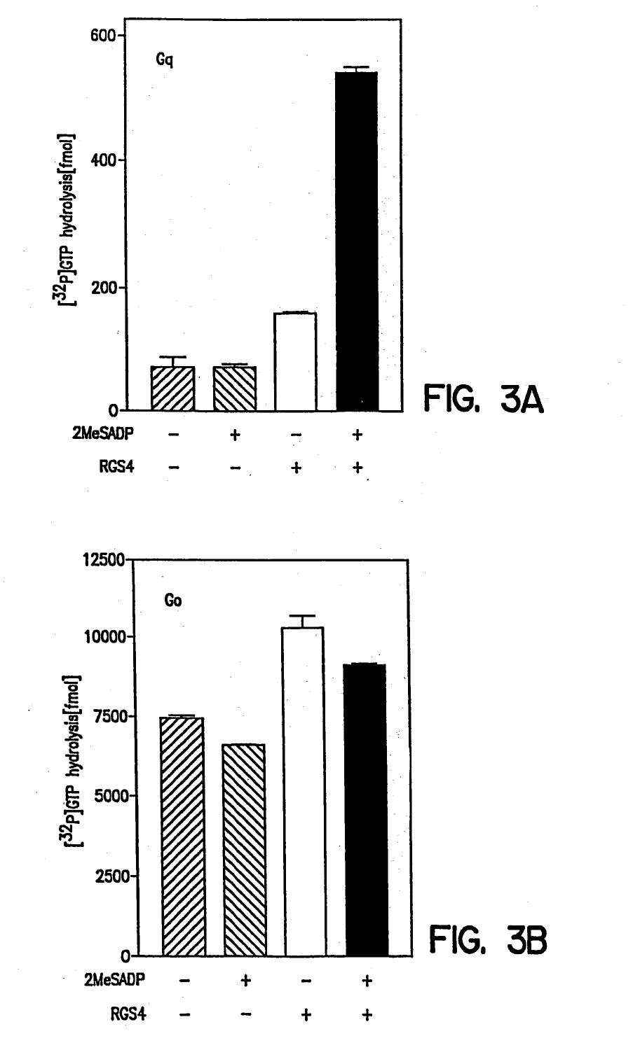

The selectivity of the P2Y₁ receptor for coupling to various G proteins, and the selectivity of RGS proteins and phospholipase C-β isoenzymes for promoting GTPase activities were studied. As illustrated in FIG. 3, the P2Y₁ receptor also couples to Gαq. Addition of carbachol to vesicles, reconstituted with purified m2-muscarinic receptors and Gαo, however, resulted in marked stimulation of GTPase activity.

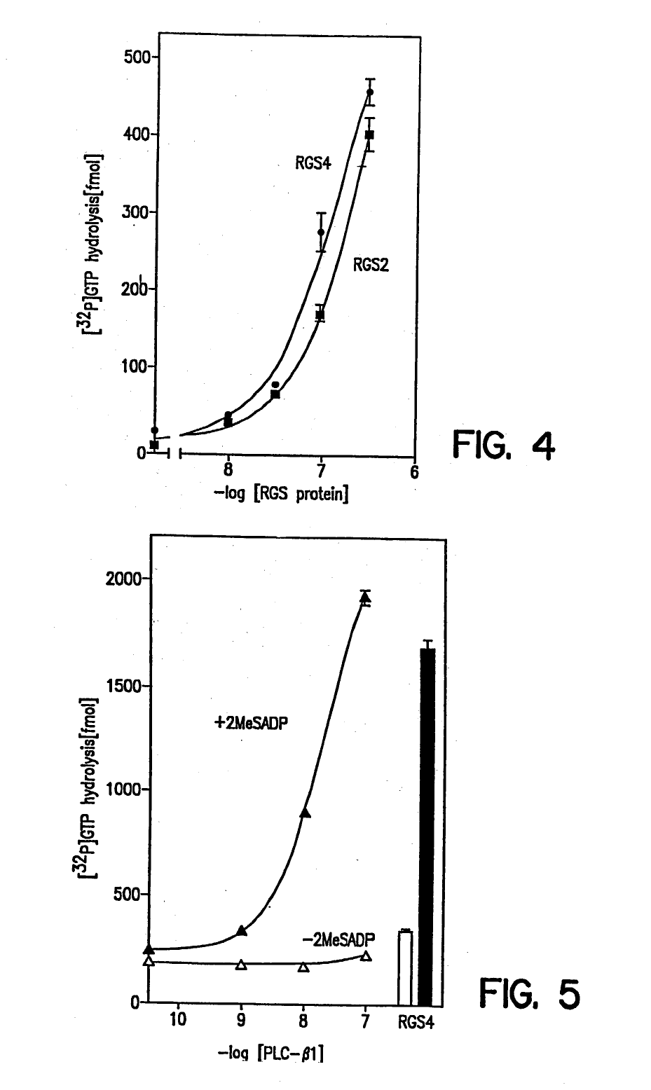

RGS2 and RGS4 were similar in their potencies and maximal activities for promotion of GTPase activity of the P2Y₁ receptor/Gαq/β1γ2 vesicles (FIG. 4). Phospholipase C-β1 also was a potent and efficacious stimulator of GSP activity of Gαq in the P2Y₁ receptor-containing vesicles (FIG. 5). The maximal stimulatory effect of phospholipase C-β1 was similar to that observed with RGS4. Turkey erythrocyte PLC-βt, which has been well studied, also stimulated GTPase activity. The potency of PLC-βt was similar to that of PLC-β1, but the maximal effect observed was somewhat lower.

### References

The references listed below as well as all references cited in the specification are incorporated herein by reference to the extent that they supplement, explain, provide a background for or teach methodology, techniques and/or compositions employed herein.

Adelman et al. (1983) DNA 2: 183.

Ausubel et al. (1992) Current Protocols in Molecular Biology, (J. Wylie & Sons, N.Y.).

Bodanszky, et al., Peptide Synthesis, John Wiley & Sons, Second Edition, 1976.

Burger and Noonan (1970) Nature 228(271): 512-15.

Crea et al. (1978) Proc. Natl. Acad. Sci. U.S.A, 75: 5765.

Eichenlaub et al. (1979) J. Bacteriol 138: 559-66

Fields et al., (!990) Int. J. Peptide Protein Res. 35: 161-214.

Hopp, U.S. Pat. No. 4,554,101.

Howell et al. (1988) Antibodies A Laboratory Manual, (Cold Spring Harbor Laboratory).

Kyte et al. (1982) J. Mol. Biol. 157: 105.

McOmie, Protective Groups in Organic Chemistry, Plenum Press, New York, (1973).

Meienhofer, Hormonal Proteins and Peptides, Vol. 2, p. 46, Academic Press (New York) (1983).

Merrifield, (1969) Adv Enzymol, 32:221-96.

Messing et al. (1981) Third Cleveland Symposium on Macromolecules and Recombinant DNA, Editor A. Walton, (Elsevier, Amsterdam).

Needleman et al., (1970) J. Mol. Biol. 48: 443.

Sambrook et al. (1992) Molecular Cloning: A Laboratory Manual (Cold Spring Harbor Laboratory Press, Cold Spring Harbor, N.Y.).

Schroder et al., The Peptides, Vol. 1, Academic Press (New York) (1965).

Smith et al., Adv. Appl. Math. 2: 482 (1981).

Steward et al., Solid Phase Peptide Synthesis, W. H. Freeman Co., San Francisco (1969).

U.S. Pat. No. 5,596,088

U.S. Pat. No. 6,043,094

U.S. Pat. No. 6,045,821

U.S. Pat. No. 6,045,822

U.S. Pat. No. 6,048,546

U.S. Pat. No. 6,063,582

Wetmur & Davidson (1968) J. Mol. Biol. 31: 349-70.

WO84/03564

WO92/21017

WO97/19170

WO99/55901

WO99/02675A1

Zimmer et al., Peptides 1992, pp. 393-394, ESCOM Science Publishers, B. V., 1993.

It will be understood that various details of the invention can be changed without departing from the scope of the invention. Furthermore, the foregoing description is for the purpose of illustration only, and not for the purpose of limitation the invention being defined by the claims.

What is claimed is:

1. A method of screening candidate substances for an ability to modulate P2Y receptor-promoted biological activity, the method comprising:
(a) establishing a test sample comprising a substantially pure P2Y receptor;
(b) contacting the test sample with a candidate substance; and
(c) measuring an interaction, effect, or combination thereof, of the candidate substance on the test sample to thereby determine the ability of the candidate substance to modulate P2Y receptor-promoted biological activity.

2. A method of screening candidate substances for an ability to modulate P2Y receptor-promoted biological activity, the method comprising:
(a) establishing replicate test and control samples that comprise a substantially pure biologically active P2Y receptor polypeptide;
(b) administering a candidate substance to test sample but not the control sample;
(c) measuring the activity of P2Y receptor-promoted biological activity in the test and the control samples; and
(d) determining that the candidate substance modulates P2Y receptor-promoted biological activity if a level of P2Y receptor-promoted activity measured for the test sample is greater or less than the level of P2Y receptor-promoted biological activity measured for the control sample.

3. A method of screening candidate substances for an ability to modulate P2Y receptor-promoted biological activity, the method comprising:
(a) establishing a control system comprising a P2Y receptor and a ligand, wherein the P2Y receptor is capable of binding to the ligand;
(b) establishing a test system comprising a P2Y receptor, a ligand, and a candidate compound;
(c) measuring a binding affinity of a P2Y receptor and a ligand in the control and the test systems; and
(d) determining that the candidate compound modulates P2Y receptor-promoted activity in a cell-free system if the binding affinity measured for the test system is less than or greater than the binding affinity measured for the control system.

4. The method of any of claims 1, 2 or 3, wherein the method is carried out in a cell-free system.

5. The method of claim 4, wherein a test sample or a control sample further comprises a vesicle comprising a P2Y receptor and a protein that normally interacts with a P2Y receptor in nature.

6. The method of claim 5, wherein the P2Y receptor is selected from the group consisting of a P2Y₁ receptor, a P2Y₂ receptor, a P2Y₄ receptor, a P2Y₆ receptor, a P2Y₁₁ receptor and combinations thereof.

7. The method of claim 5, wherein the protein that normally interacts with a P2Y receptor in nature is a G protein.

8. The method of claim 7, wherein the G protein is selected from the group including but not limited to Gₐα, G_{q}β, Gₐγ, G₁₁α, G_{12/13}α, G_{12/13}β, G_{12/13}γ, Gᵢα, Gᵢβ, Gᵢγ, Gₛα, Gₛ,β,Gₛγ, Gα₁₄, Gα₁₆, Gβγ dimers, and combinations thereof.

9. The method of claim 5, wherein the protein that normally interacts with a P2Y receptor in nature is substantially pure.

10. The method of claim 5, wherein a test sample or a control sample further comprises a ligand for the P2Y receptor or for the protein that normally interacts with a P2Y receptor in nature.

11. The method of claim 10, wherein the ligand is selected from the group consisting NTP, NDP, a RGS protein, an agonist, an antagonist, and combinations thereof.

12. The method of claim 11, wherein the RGS protein is selected from the group consisting of RGS1, RGS2, RGS4, RGS16 and combinations thereof.

13. The method of claim 10, wherein the ligand, the P2Y receptor, the protein that normally interacts with a P2Y receptor, or combination thereof is detectably labeled.

14. The method of claim 13, wherein the label is a radioactive moiety, a fluorescent moiety, a chemiluminescent moiety, or combination thereof.

15. The method of claim 14, wherein the radioactive moiety is selected from the group consisting of ³H, ³²P, ³⁵s, ¹⁴C, ¹²⁵I and combinations thereof.

16. The method of claim 14, wherein the fluorescent moiety is selected from the group consisting of a near-infrared fluorescent dye, dinitrophenyl, fluorescein and derivatives thereof, rhodamine, derivatives of rhodamine, phycoerythrin, phycocyanin, allophycocyanin, o-phthaldehyde and fluorescamine, Texas red, Rhodamine green, Oregon green, Cascade blue, phycoerythrin, CY3, CY5, CY2, CY7, coumarin, infrared 40, MR 200, IRD 40, green fluorescent protein and combinations thereof.

17. The method of claim 14, wherein the chemiluminescent moiety is selected from the group consisting of luminol, isoluminol, theromatic acridinium ester, imidazole, acridinium salt and oxalate ester, luciferin, luciferase and aequorin and combinations thereof.

18. The method of claim 14, wherein the binding affinity is assessed by comparing an amount of bound labeled ligand to an amount of unbound labeled ligand.

19. The method of claim 18, wherein the bound and unbound labeled ligands are separated by contacting a test sample with a separation matrix.

20. The method of claim 19, wherein the separation matrix comprises activated charcoal.

21. The method of claim 14, wherein a detectable signal is generated from resonant interaction between two energy emitting label moieties.

22. The method of any of claims 1, 2 or 3, wherein the P2Y receptor-promoted biological activity is selected from the group consisting of hydrolysis of NTP molecules to NDP molecules, formation of NTP molecules from NDP molecules, modulation of intracellular calcium levels, modulation of phospholipase C activity, modulation of adenylate cyclase activity, translocation of RhoA to membranes, formation of a network of stress fibers, phosphorylation of myosin light chains, cell differentiation modulation of NTPase activity, shape change in platelets and combinations thereof.

23. The method of any of claims 1, 2 or 3, wherein the method is carried out in at least one well of a multi-well plate.

24. The method of any of claims 1, 2 or 3, further comprising screening a plurality of candidate substances simultaneously.

25. The method of any of claims 24, wherein the method is carried out in multiple wells of a multi-well plate.

26. A cell-free system for the study of P2Y receptors comprising:
(a) a P2Y receptor;
(b) a protein that normally interacts with the P2Y receptor in nature; and
(c) a vesicle.

27. The system of claim 26, wherein the P2Y receptor is substantially pure.

28. The system of claim 26, wherein the P2Y receptor is selected from the group consisting of a P2Y₁ receptor, a P2Y₂ receptor, a P2Y₄ receptor, a P2Y₆ receptor, a P2Y₁₁ receptor and combinations thereof.

29. The system of claim 26, wherein the protein that normally interacts with a P2Y receptor in nature is substantially pure.

30. The system of claim 26, wherein the protein that normally interacts with a P2Y receptor in nature is a G protein.

31. The system of claim 30, wherein the G protein is selected from the group including but not limited to G_{q}α, G_{q}β, G_{q}γ, G¹¹α, G_{12/13}α, G_{12/13}β, G_{12/13}γ, Gᵢα, Gᵢβ, Gᵢγ, Gₛα, Gₛβ,Gₛγ, G_{α}₁₄, Gα₁₆, Gβγ dimers, and combinations thereof.

32. The system of claim 26, further comprising a ligand for the P2Y receptor or for the protein that normally interacts with a P2Y receptor in nature.

33. The system of claim 32, wherein the ligand is selected from the group consisting NTP, NDP, a RGS protein, an agonist. an antagonist, and combinations thereof.

34. The system of claim 33, wherein the RGS protein is selected from the group consisting of RGS1, RGS2, RGS4, RGS16 and combinations thereof.

35. The system of claim 32, wherein the ligand, the P2Y receptor, the protein that normally interacts with a P2Y receptor, or combination thereof is detectably labeled.

36. The system of claim 35, wherein the label is a radioactive moiety, a fluorescent moiety, a chemiluminescent moiety, or combination thereof.

37. The system of claim 36, wherein the radioactive moiety is selected from the group consisting of ³H, ³²P, ³⁵S, ¹⁴C, ¹²⁵I and combinations thereof.

38. The system of claim 36, wherein the fluorescent moiety is selected from the group consisting of a near-infrared fluorescent dye, dinitrophenyl, fluorescein and derivatives thereof, rhodamine, derivatives of rhodamine, phycoerythrin, phycocyanin, allophycocyanin, o-phthaldehyde and fluorescamine, Texas red, Rhodamine green, Oregon green, Cascade blue, phycoerythrin, CY3, CY5, CY2, CY7, coumarin, infrared 40, MR 200, IRD 40, green fluorescent protein and combinations thereof.

39. The system of claim 36, wherein the chemiluminescent moiety is selected from the group consisting of luminol, isoluminol, theromatic acridinium ester, imidazole, acridinium salt and oxalate ester, luciferin, luciferase and aequorin and combinations thereof.

40. A method of producing a cell-free system for the assay of P2Y receptor-promoted activity, the method comprising:
(a) purifying a P2Y receptor;
(b) purifying at least one protein that normally interacts with the P2Y receptor in nature;
(c) reconstituting the P2Y receptor into a vesicle; and
(d) reconstituting at least one protein that normally interacts with the P2Y receptor in nature into the vesicle to thereby produce the cell-free system.

41. The method of claim 40, wherein the P2Y receptor is selected from the group consisting of a P2Y₁ receptor, a P2Y₂ receptor, a P2Y₄ receptor, a P2Y₆ receptor, a P2Y₁₁ receptor and combinations thereof.

42. The method of claim 40, wherein the P2Y receptor is expressed in an in vivo or in vitro expression system.

43. The method of claim 41, wherein the expression system further comprises a recombinant vector comprising a nucleic acid sequence encoding a P2Y receptor.

44. The method of claim 43, wherein the recombinant vector further comprises:
(a) a sequence of genomic viral DNA showing affinity for a host cell and possessing the ability to infect said host cell;
(b) a nucleic acid sequence encoding a P2Y receptor operatively linked to the sequence of genomic viral DNA, wherein the operatively-linked P2Y receptor is expressed in said host cell following infection of the cell; and
(c) a selectable marker.

45. The method of claim 44, wherein the sequence of genomic viral DNA is baculoviral DNA.

46. The method of claim 43, further comprising transfecting a cell with the recombinant vector under conditions suitable for the expression of the P2Y receptor to thereby produce a P2Y receptor.

47. The method of claim 46, wherein the cell is a prokaryotic cell or is a eukaryotic cell.

48. The method of claim 47, wherein the cell is an insect cell.

49. The method of claim 42, wherein the P2Y receptor has a sequence of at least six histidine residues at the N-terminal end of the P2Y receptor protein.

50. The method of claim 42, wherein the P2Y receptor has a sequence of at least six histidine residues at the C-terminal end of the P2Y receptor protein.

51. The method of claim 42, wherein the P2Y receptor comprises a FLAG® epitope at the N terminal end of the P2Y receptor protein.

52. The method of claim 42, wherein the P2Y receptor expresses a FLAG® epitope at the C terminal end of the P2Y receptor protein.

53. The method of claim 49 or 50, wherein the P2Y receptor is purified by passing the receptor over a residue comprising nickel atoms.

54. The method of claim 51 or 52, wherein the P2Y receptor is purified by binding the receptor to a detectable anti-FLAG® antibody and isolating the complex.

55. The method of claim 40, wherein the proteins that normally interact with the P2Y receptor in nature are expressed in an in vitro or in vivo expression system.

56. The method of claim 55, wherein the protein that normally interacts with a P2Y receptor in nature is a G protein.

57. The method of claim 7, wherein the G protein is selected from the group including but not limited to G_{q}α, G_{q}β, G_{q}γ, G₁₁α, G_{12/13}α, G_{12/13}β, G_{12/13}γ, Gᵢα, Gᵢβ, Gᵢγ, Gₛα, Gₛβ,Gₛγ, Gβγ dimers, and combinations thereof.

58. The method of claim 40, further comprising adding a ligand to the cell-free system.

59. The method of claim 58, wherein the ligand is selected from the group consisting NTP, NDP, a RGS protein, an agonist, an antagonist, and combinations thereof.

60. The method of claim 59, wherein the RGS protein is selected from the group consisting of RGS1, RGS2, RGS4, RGS16 and combinations thereof.

61. The method of claim 58, wherein the ligand, the P2Y receptor, the protein that normally interacts with a P2Y receptor, or combination thereof is detectably labeled.

62. The method of claim 61, wherein the label is a radioactive moiety, a fluorescent moiety, a chemiluminescent moiety, or combination thereof.

63. The method of claim 62, wherein the radioactive moiety is selected from the group consisting of ³H, ³²P, ³⁵s, ¹⁴C, ¹²⁵I and combinations thereof.

64. The method of claim 62, wherein the fluorescent moiety is selected from the group consisting of a near-infrared fluorescent dye, dinitrophenyl, fluorescein and derivatives thereof, rhodamine, derivatives of rhodamine, phycoerythrin, phycocyanin, allophycocyanin, o-phthaldehyde and fluorescamine, Texas red, Rhodamine green, Oregon green, Cascade blue, phycoerythrin, CY3, CY5, CY2, CY7, coumarin, infrared 40, MR 200, IRD 40, green fluorescent protein and combinations thereof.

65. The method of claim 62, wherein the chemiluminescent moiety is selected from the group consisting of luminol, isoluminol, theromatic acridinium ester, imidazole, acridinium salt and oxalate ester, luciferin, luciferase and aequorin and combinations thereof.

66. A method of producing a cell-free system for the assay of P2Y receptor-promoted activity, the method comprising:
(a) purifying a P2Y receptor; and
(b) reconstituting the P2Y receptor into a vesicle.

67. A cell-free system for the study of P2Y receptors comprising:
(a) a P2Y receptor; and
(b) a vesicle.


SEQUENCE LISTING (1 sequence)

SEQ ID NO: 1 - protein, 8 aa, Artificial Sequence
  Description of Artificial Sequence  immunotag epitope
DYKDDDDK